(19) United States
(12) Patent Application Publication
(10) Pub. No.: US 2003/0220604 A1
(43) Pub. Date: Nov. 27, 2003

(54) Al-Anazi ventriculo uterine shunt

(76) Inventor: Abdul Rahman Al-Anazi, Alkhobar (SA)

Correspondence Address: DONALD W. MEEKER, 924 EAST OCEAN FRONT #E, NEWPORT BEACH, CA, 92661, US

(21) Appl. No.: 10/156,353

(22) Filed: May 26, 2002

(51) Int. Cl. A61M 5/00; A61M 25/00

(52) U.S. Cl. 604/9; 604/8; 604/264; 604/523

(57) ABSTRACT

A short catheter of 25-30 mm has a longitudinal central opening having an internal diameter of at least 1.25 mm. Paired wings adjacent to a proximal end secure the shunt to the skull. Four openings, with two on each side, admit cerebral fluid from the ceberal cavity to the central opening and up to a one-way valve at the proximal end of the outside of the the skull. The valve lets fluid out into the uterine cavity of the mother and prevents backflow of fluid into the cerebral cavity of the fetus.

### BACKGROUND OF THE INVENTION

1. Field of the Invention

The present invention relates to hydrocephalic shunts and in particular to an in utero hydrocephalic shunt having a short length and large opening to prevent blockage, having a calibrated one-way valve to allow adequate drainage and having a minimal outer protrusion to prevent complications for the mother.

2. Description of the Prior Art

Hydrocephalism is a tragic disease in which a child in the uterus suffers from an excess of fluid in the brain cavity which enlarges the head and causes brain damage, so that the child is born with an enlarged distorted head and lost mental capacity due to brain damage. This disease is detectable in utero and should be treated before birth to prevent the advancement of the disease and severe damage caused before birth.

There are examples of in utero treatment and several inventions have been patented related to such treatment, but have drawbacks related to clogging and protruding too far out of the skull of the fetus.

U.S. Pat. No. 4,475,898, issued Oct. 9, 1984 to Brodner, shows a fetal ventriculo-amniotic shunt with two flanges or inflatable cuffs located midway serving to retain the device in place with one inside and one outside the skull. The device may include a miter valve near the inner end adjacent to the openings for the CSF or a slit valve at the outer end emptying into the amniotic fluid. The shunt is long (2.8 inches) and narrow (0.052 inches outside diameter and 0.027 inches inside diameter).

U.S. Pat. No. 4,474,569, issued Oct. 2, 1984 to Newkirk, provides an ante natal micro shunt (0.047 inches outside diameter) with expandable wing anchors, one inside and one outside the skull. It includes a check valve near the midpoint and a gripping element on the outer end. The device is long and narrow and with the wing anchors retracted the device may be implanted with a hollow surgical needle using ultrasonography as a guidance aid.

U.S. Pat. No. 4,413,985, issued Nov. 8, 1983 to Wellner, claims a hydrocephalic ante natal vent for intra-uterine treatment using a hydrocephalic drainage valve having a conical head a spring biased ball check valve adjusted by a hollow set screw all inside a relatively short hollow tube with external threads for gripping the skull adjacent to an overhanging cap for contacting the outside of the skull. The device is short but quite narrow inside (the ball is only 0.047 inches in diameter).

What is needed is a secure in utero hydrocephalic shunt which does not clog and does not protrude far out of the skull of the fetus.

### SUMMARY OF THE INVENTION

An object of the present invention is to provide an in utero hydrocephalic shunt which has a sufficiently large internal diameter such that is it not subject to clogging.

Another object of the present invention is to provide an in utero hydrocephalic shunt which has only a small somewhat rounded valve protruding from the head of the fetus.

One more object of the present invention is to provide an in utero hydrocephalic shunt with a double set of protruding side wings for engaging the skull so that the shunt does not move either into or out of the skull once in place.

In brief the in utero shunt of the present invention has a short length of 25-30 mm sufficiently long to penetrate into the skull of the fetus but not protrude outside of the skull into the uterus very far and a relatively large internal diameter of 1.25 mm to prevent internal obstruction and clogging.

A pair of external flexible wings at an outer end of the shunt spread a sufficient distance on the outside of the skull of the fetus (12 mm from tip to tip) to prevent the shunt from moving further into the skull once in place. A pair of inner flexible wings extend out from the shunt body a sufficient distance (7 mm from tip to tip) to prevent the shunt from pulling out of the skull, but due to their flexibility the shorter wings are easy to insert into the skull while the outer wings stop the insertion.

A one-way valve, preferably a low pressure silicone elastomer Cruciform Valve is positioned outside of the skull to permit the internal portion of the shunt to be only the necessary diameter (2 mm outer diameter, 1.25 mm interior diameter) to prevent clogging. The external valve is a relatively small rounded protrusion on the end of the shunt positioned externally of the skull.

An advantage of the present invention is that it functions well to maintain the desired cranial pressure with no blockage of the shunt and no backflow through the valve.

Another advantage of the present invention is that a minimal and rounded external protrusion causes little or not problems in affecting the interior walls of the uterus of the mother.

An additional advantage of the present invention is that it stays securely implanted in the skull of the fetus.

### BRIEF DESCRIPTION OF THE DRAWINGS

These and other details of my invention will be described in connection with the accompanying drawings, which are furnished only by way of illustration and not in limitation of the invention, and in which drawings:

FIG. 1 is a cross-sectional view of the invention in place inserted into the cranial cavity of the fetus with only the one-way valve protruding into the uterus of the mother.

### BEST MODE FOR CARRYING OUT THE INVENTION

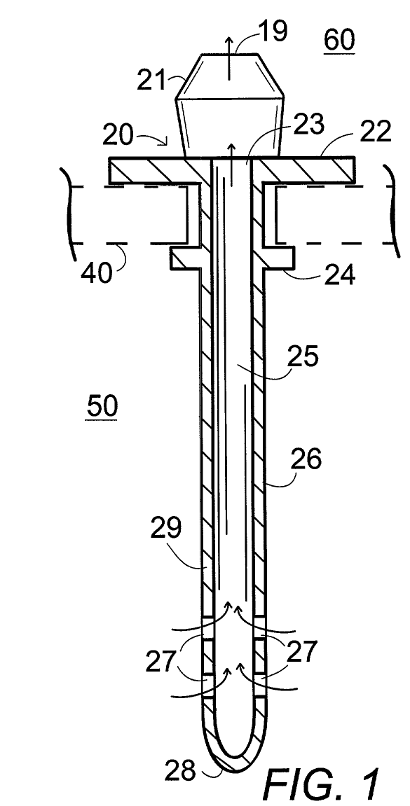

In FIG. 1, an in utero hydrocephalic shunt comprises a ventricular catheter comprising an elongated tubular structure 26 capable of being inserted within a cranial cavity 50 of a fetus, the ventricular catheter having a thin wall 29 with a central longitudinal opening 25 along its length and at least one (preferably four) ventricular openings 27 through the wall at a distal ventricular end 28 having a rounded point for insertion within a brain cavity 50 of a fetus. The ventricular openings 27 are preferably positioned with two on each side to avoid blockage and they communicate with the central longitudinal opening 25 to admit cerebral fluid (flow shown by arrows) therethrough from the brain cavity 50 into the central longitudinal opening 25. The catheter comprises a straight anti-block ventricular catheter and the length of the catheter is 25-30 mm for adequate penetration into the cranial cavity of the uterus to drain fluid. The inner diameter forming the central longitudinal opening 25 is at least 1.25 mm to prevent blockage and the outer diameter of the catheter is at least 2 mm.

The ventricular catheter further comprises external protruding retention means extending from the wall 29, comprising a first pair of protrusions or first side wings 22 extending outwardly from the catheter outside of the skull 40 and in contact with an exterior of the skull 40 and a second pair of protrusions or second side wings 24 extending outwardly from the catheter inside of the skull and in contact with the interior of the skull 40. The protrusions secure the shunt 20 in place extending between the cerebral cavity 50 and the uterine cavity 60 and prevent movement of the shunt and retain the shunt within the brain cavity by contact with a skull 40 of the fetus.

The second pair of protrusions or second side wings 24 are flexible and capable of flexing for insertion of the catheter into the cerebral cavity and they measure at most 7 mm from outer end to outer end. The first pair of protrusions or first side wings 22 measure at least 12 mm from outer end to outer end. The first and second pair of protrusions are separated by 5 mm to accommodate the skull 40 of the fetus therebetween.

The ventricular catheter has a proximal opening 23 through the wall at a proximal end, the proximal opening communicating with the central longitudinal opening 25.

A one-way valve 21 is attached to the ventricular catheter 26 at the proximal end so that the valve is positioned with a valve opening 19 into a uterine cavity 50 of a mother of the fetus and the proximal opening 23 is in communication with the valve so that cerebral fluid passing through the central longitudinal opening is capable of passing through the valve out into the uterine cavity of the mother of the fetus (flow shown by arrows) and the valve is capable of blocking fluid from passing back into the central longitudinal opening from the uterine cavity 60. The valve 21 preferably comprises a low pressure silicone elastomer Cruciform Valve.

Positioning the valve 21 on the exterior presents only a small somewhat rounded protrusion into the uterine cavity 60 to minimize any problems by contact with the uterine wall and allows the catheter 26 positioned within the cerebral cavity 50 of the fetus to have a central longitudinal opening 25 of optimum diameter (at least 1.25 mm) to prevent clogging.

The entire catheter may be molded of flexible plastic and the device is packaged in a sterile environment.

It is understood that the preceding description is given merely by way of illustration and not in limitation of the invention and that various modifications may be made thereto without departing from the spirit of the invention as claimed.

What is claimed is:

1. An Al-Anazi ventriculo uterine shunt comprising:
a ventricular catheter comprising an elongated tubular structure capable of being inserted within a cranial cavity of a fetus, the ventricular catheter having a thin wall with a central longitudinal opening along its length and at least one ventricular opening through the wall at a distal ventricular end insertable within a brain cavity of a fetus, the at least one ventricular opening communicating with the central longitudinal opening and the at least one ventricular opening capable of admitting cerebral fluid therethrough from the brain cavity into the central longitudinal opening and the ventricular catheter further comprising external protruding retention means extending from the wall, the retention means capable of retaining the shunt within the brain cavity by contact with a skull of the fetus, the ventricular catheter having a proximal opening through the wall at a proximal end, the proximal opening in communication with the central longitudinal opening;
a one-way valve attached to the ventricular catheter at the proximal end so that the valve is positioned with a valve opening into a uterine cavity of a mother of the fetus and the proximal opening is in communication with the valve so that cerebral fluid passing through the central longitudinal opening is capable of passing through the valve out into the uterine cavity of the mother of the fetus and the valve is capable of blocking fluid from passing back into the central longitudinal opening from the uterine cavity.

2. The Al-Anazi ventriculo uterine shunt of claim 1 wherein the retention means comprises a first pair of protrusions extending outwardly from the catheter outside of the skull and in contact with an exterior of the skull and a second pair of protrusions extending outwardly from the catheter inside of the skull and in contact with the interior of the skull, the protrusions capable of securing the shunt in place extending between the cerebral cavity and the uterine cavity and capable of preventing movement of the shunt.

3. The Al-Anazi ventriculo uterine shunt of claim 2 wherein the second pair of protrusions are flexible and capable of flexing for insertion of the catheter into the cerebral cavity.

4. The Al-Anazi ventriculo uterine shunt of claim 2 wherein the second pair of protrusions measure at most 7 mm from outer end to outer end.

5. The Al-Anazi ventriculo uterine shunt of claim 2 wherein the first and second pair of protrusions are separated by 5 mm.

6. The Al-Anazi ventriculo uterine shunt of claim 2 wherein the first pair of protrusions measure at least 12 mm from outer end to outer end.

7. The Al-Anazi ventriculo uterine shunt of claim 1 wherein the valve is positioned at the proximal end of the catheter external to the skull of the fetus.

8. The Al-Anazi ventriculo uterine shunt of claim 7 the valve comprises a low pressure silicone elastomer Cruciform Valve.

9. The Al-Anazi ventriculo uterine shunt of claim 1 wherein the catheter comprises a straight anti-block ventricular catheter.

10. The Al-Anazi ventriculo uterine shunt of claim 1 wherein the length of the catheter is 25-30 mm.

11. The Al-Anazi ventriculo uterine shunt of claim 1 wherein the inner diameter of the catheter forming the width of the central longitudinal opening of the catheter is at least 1.25 mm.

12. The Al-Anazi ventriculo uterine shunt of claim 1 comprising at least four ventricular openings through the wall of the catheter at the ventricular distal end of the catheter with at least two ventricular openings on each of two opposing sides of the catheter.
